(19) United States
(12) Patent Application Publication
(10) Pub. No.: US 2023/0080988 A1
(43) Pub. Date: Mar. 16, 2023

(54) CHAOTROPIC AGENTS FOR REDUCING FORMATION OF DOUBLE-STRANDED RNA

(71) Applicant: ULTRAGENYX PHARMACEUTICAL INC., Novato, CA (US)

(72) Inventors: Xijun PIAO, South San Francisco, CA (US); Wayne CHANG, Milpitas, CA (US); Benjamin E. LINDENMUTH, San Rafael, CA (US); Xiaowei WANG, Hillsborough, CA (US)

(73) Assignee: ULTRAGENYX PHARMACEUTICAL INC., Novato, CA (US)

(21) Appl. No.: 17/797,086

(22) PCT Filed: Feb. 4, 2021
(86) PCT No.: PCT/US2021/016613

§ 371 Date: Aug. 2, 2022

Related U.S. Application Data
(60) Provisional application No. 62/971,777, filed on Feb. 7, 2020.

(51) Int. Cl. C07H 21/02; C07H 1/00

(52) U.S. Cl. CPC C07H 21/02; C07H 1/00

(57) ABSTRACT

The present disclosure provides methods of reducing, minimizing, or inhibiting the formation of double-stranded ribonucleic acid (dsRNA) during the preparation of ribonucleic acid (RNA), such as messenger ribonucleic acid (mRNA), by adding at least one chaotropic agent to a starting reaction mixture. The present disclosure provides methods of reducing, minimizing, or inhibiting intramolecular base-pairing within a ribonucleic acid (RNA) transcript and/or intermolecular base-pairing between an RNA transcript and deoxyribonucleic acid (DNA) or another RNA during the preparation of ribonucleic acid (RNA), by adding at least one chaotropic agent to a starting reaction mixture.

### CROSS-REFERENCE TO RELATED APPLICATIONS

This application is being filed on Feb. 4, 2021, as a PCT International Patent Application and claims priority to and the benefit of U.S. Provisional Patent Application Ser. No. 62/971,777, filed Feb. 7, 2020, the disclosure of which is hereby incorporated by reference in its entirety.

### FIELD OF THE DISCLOSURE

The present disclosure relates to the use of chaotropic agents to minimize the formation of double-stranded ribonucleic acid during the preparation of messenger ribonucleic acid.

### INTRODUCTION

Messenger ribonucleic acid (mRNA) is the product of transcription of deoxyribonucleic acid (DNA) and serves as the template for translation to generate proteins. The genetic code of the mRNA specifies the amino acid sequence for protein synthesis. Transcription generally occurs inside the cells of an organism. The synthesis of larger, even mass, amounts of mRNA can be carried out in vitro.

mRNA-based vaccines can induce cell-mediated immunity important for cancer vaccines through MHC class I proteins, because mRNA can direct the synthesis of antigenic proteins directly in the cell. Furthermore, mRNA itself can be developed as a therapeutic agent capable of being translated into protein in a subject's cells to prevent, treat, and/or cure disease. For therapeutic use of mRNA, it is critical that the synthesis procedure generates mRNA as a single molecular species in a highly pure form. Scalability is also important.

In vitro transcription (IVT) of mRNA is gaining importance in the synthesis of therapeutic mRNA products, and the purity of the mRNA product is critical to its use in a therapeutic context. One major contaminant in the IVT-produced mRNA is double-stranded RNA (dsRNA), identified as a trigger of cellular immune response. Unfortunately, dsRNA is not sufficiently removed from IVT-produced mRNA using such standard purification methods as LiCl, alcohol-based precipitation, size exclusion chromatography, ion exchange chromatography, and silica matrix-based purification. And while ion pair reversed-phase high-performance liquid chromatography (RP-HPLC) has been used for purification, it is neither scalable nor affordable, and the acetonitrile eluent is very toxic. Hydroxyapatite (CHT) has also been used to remove dsRNA selectively. However, both RP-HPLC and CHT need to be performed at elevated temperatures to effectively remove dsRNA, and such elevated temperature is detrimental to the integrity of long mRNA. Recently, RNase III and cellulose powder have been used to selectively digest dsRNA and absorb dsRNA in the presence of desired mRNA, respectively. However, controlling the activity of RNase III, removal of partially degraded mRNA, potential leachable impurities from cellulose, scalability, and loss of intact mRNA on cellulose raise concern.

Because existing purification strategies are costly, impractical for scalability, and suffer other limitations, there is a need for improved methods for preparing mRNA products that contain minimal transcription errors, can be produced from a laboratory scale to a mass scales, and are highly pure. The present disclosure addresses this need by providing methods for the reduction of dsRNA formation during RNA synthesis through the use of one or more chaotropic agents.

### SUMMARY OF THE INVENTION

In a first aspect, the disclosure provides a method of reducing, minimizing, or inhibiting the formation of double-stranded ribonucleic acid (dsRNA) during the preparation of ribonucleic acid (RNA), comprising adding at least one chaotropic agent to a starting reaction mixture.

In another aspect, the disclosure provides a method of reducing, minimizing, or inhibiting intramolecular base-pairing within a ribonucleic acid (RNA) transcript and/or intermolecular base-pairing between an RNA transcript and deoxyribonucleic acid (DNA) or another RNA during the preparation of ribonucleic acid (RNA), comprising adding at least one chaotropic agent to a starting reaction mixture.

In still another aspect, the disclosure provides a method of reducing, minimizing, or inhibiting the formation of double-stranded ribonucleic acid (dsRNA) during in vitro transcription, comprising adding at least one chaotropic agent to an in vitro transcription reaction mixture.

In yet another aspect, the disclosure provides a method of reducing, minimizing, or inhibiting intramolecular base-pairing within a ribonucleic acid (RNA) transcript and/or intermolecular base-pairing between an RNA transcript and deoxyribonucleic acid (DNA) or another RNA during in vitro transcription, comprising adding at least one chaotropic agent to an in vitro transcription reaction mixture.

In certain embodiments of a method according to the disclosure, the transcription yields ribonucleic acid (RNA).

In certain embodiments of a method according to the disclosure, the RNA is selected from the group consisting of messenger RNA (mRNA), ribosomal RNA (rRNA), transfer RNA (tRNA), and microRNA (miRNA). In further embodiments of a method according to the disclosure, the RNA is messenger RNA (mRNA).

In certain embodiments of a method according to the disclosure, the amount or yield of RNA is not significantly reduced by the addition of the at least one chaotropic agent. In further embodiments, the amount or yield of RNA is not reduced more than about 75% by the addition of the at least one chaotropic agent. In still further embodiments, the amount or yield of RNA is not reduced more than about 50%, more than about 25%, more than about 20%, more than about 15%, more than about 10%, or more than about 5% by the addition of the at least one chaotropic agent.

In certain embodiments of a method according to the disclosure, the at least one chaotropic agent is selected from the group consisting of urea, formamide, sodium salicylate, ethanol, sodium perchlorate, arginine, n-butanol, thiourea, and 2-propanol.

In certain embodiments of a method according to the disclosure, at least two (i.e., two, three, four, or more) chaotropic agents are used in combination to reduce the formation of dsRNA. For instance, in some embodiments, a first chaotropic agent (e.g., urea) may be used in combination with a second chaotropic agent (e.g., formamide) for reducing dsRNA. In embodiments where at least two chaotropic agents are used, the chaotropic agents may be added to the reaction mixture simultaneously, separately, or sequentially.

In certain embodiments of a method according to the disclosure, the at least one chaotropic agent is urea. In additional embodiments, the urea is at a concentration of from about 0.1M to about 1.6M.

In certain embodiments of a method according to the disclosure, the at least one chaotropic agent is formamide. In additional embodiments, the formamide is at a concentration of from about 0.1M to about 2.8M.

In certain embodiments of a method according to the disclosure, the at least one chaotropic agent is sodium perchlorate. In additional embodiments, the sodium perchlorate is at a concentration of from about 0.01M to less than about 0.15M.

In certain embodiments of a method according to the disclosure, the at least one chaotropic agent is sodium salicylate. In additional embodiments, the sodium salicylate is at a concentration of from about 0.005M to less than about 0.1M.

In certain embodiments of a method according to the disclosure, the at least one chaotropic agent is arginine. In additional embodiments, the arginine is at a concentration of from about 0.01M to less than about 0.2M.

In certain embodiments of a method according to the disclosure, the at least one chaotropic agent is ethanol. In additional embodiments, the ethanol is at a concentration of from about 0.4M to less than about 1.6M.

In certain embodiments of a method according to the disclosure, the reaction mixture is on the order of microliters to the order of milliliters to the order of liters to the order of thousands of liters.

In certain embodiments of a method according to the disclosure, the presence or absence of dsRNA is determined using denaturing gel electrophoresis, native gel electrophoresis, anti-dsRNA antibody, intact mass spectrometry, and/or controls for dsRNA.

In one aspect, the disclosure provides a composition comprising RNA prepared according to a method disclosed herein, wherein the composition is substantially free of dsRNA. In one embodiment, the composition prepared according to a method disclosed herein comprises mRNA and is substantially free of dsRNA. In certain embodiments of a composition according to the disclosure, the presence or absence of dsRNA is determined using denaturing gel electrophoresis, native gel electrophoresis, anti-dsRNA antibody, intact mass spectrometry, and/or controls for dsRNA.

In one aspect, the disclosure provides an in vitro transcription reaction mixture comprising at least one chaotropic agent for use in the preparation of ribonucleic acid (RNA).

In certain embodiments of a reaction mixture according to the disclosure, the RNA is substantially free of double-stranded ribonucleic acids (dsRNA).

In certain embodiments of a reaction mixture according to the disclosure, the RNA is messenger RNA (mRNA).

In certain embodiments of a reaction mixture according to the disclosure, the at least one chaotropic agent is selected from the group consisting of urea, formamide, guanidinium chloride, guanidine hydrochloride, sodium salicylate, dimethylsulfoxide (DMSO), ethanol, phenol, sodium dodecyl sulfate (SDS), sodium perchlorate, propylene glycol, arginine, n-butanol, thiourea, and 2-propanol.

In certain embodiments of a reaction mixture according to the disclosure, the at least one chaotropic agent is urea. In additional embodiments, the urea is at a concentration of from about 0.1M to about 1.6M.

In certain embodiments of a reaction mixture according to the disclosure, the at least one chaotropic agent is formamide. In additional embodiments, the formamide is at a concentration of from about 0.1M to about 2.8M.

In certain embodiments of a reaction mixture according to the disclosure, the at least one chaotropic agent is sodium perchlorate. In additional embodiments, the sodium perchlorate is at a concentration of from about 0.01M to less than about 0.15M.

In certain embodiments of a reaction mixture according to the disclosure, the at least one chaotropic agent is sodium salicylate. In additional embodiments, the sodium salicylate is at a concentration of from about 0.005M to less than about 0.1M.

In certain embodiments of a reaction mixture according to the disclosure, the at least one chaotropic agent is arginine. In additional embodiments, the arginine is at a concentration of from about 0.01M to less than about 0.2M.

In certain embodiments of a reaction mixture according to the disclosure, the at least one chaotropic agent is ethanol. In additional embodiments, the ethanol is at a concentration of from about 0.4M to less than about 1.6M.

In certain embodiments of a reaction mixture according to the disclosure, the presence or absence of dsRNA is determined using denaturing gel electrophoresis, native gel electrophoresis, anti-dsRNA antibody, intact mass spectrometry, and/or controls for dsRNA.

In an additional aspect, the disclosure provides the use of at least one chaotropic agent in a method of reducing, minimizing, or inhibiting the formation of double-stranded ribonucleic acid (dsRNA) during the preparation of ribonucleic acid (RNA), comprising adding the at least one chaotropic agent to a starting reaction mixture.

In another aspect, the disclosure provides the use of at least one chaotropic agent in a method of reducing, minimizing, or inhibiting intramolecular base-pairing within a ribonucleic acid (RNA) transcript and/or intermolecular base-pairing between an RNA transcript and deoxyribonucleic acid (DNA) or another RNA during the preparation of ribonucleic acid (RNA), comprising adding the at least one chaotropic agent to a starting reaction mixture.

In still another aspect, the disclosure provides the use of at least one chaotropic agent in a method of reducing, minimizing, or inhibiting the formation of double-stranded ribonucleic acid (dsRNA) during in vitro transcription, comprising adding the at least one chaotropic agent to an in vitro transcription reaction mixture.

In yet another aspect, the disclosure provides the use of at least one chaotropic agent in a method of reducing, minimizing, or inhibiting intramolecular base-pairing within a ribonucleic acid (RNA) transcript and/or intermolecular base-pairing between an RNA transcript and deoxyribonucleic acid (DNA) or another RNA during in vitro transcription, comprising adding the at least one chaotropic agent to an in vitro transcription reaction mixture

Other embodiments will become apparent from a review of the ensuing detailed description.

### BRIEF DESCRIPTION OF THE DRAWINGS

FIGS. 1A and 1B show (FIG. 1A) the mRNA yield for in vitro transcription (IVT) including different concentrations of urea (lanes labeled according to Table 2), and (FIG. 1B) a J2 mAb dot blot to detect dsRNA for the same IVT conditions as identified above. Each dot contains 1000 ng mRNA, and comparative dots are shown to the right (10 ng and 2 ng dsRNA control sample).

FIGS. 2A, 2B, 2C, and 2D show (FIGS. 2A and 2B) the mRNA yield for in vitro transcription (IVT) including different concentrations of formamide (lanes labeled according to Table 3), and (FIGS. 2C and 2D) two J2 mAb dot blots to detect dsRNA for the same IVT conditions as identified above. Each dot contains 1000 ng mRNA, and comparative dots are shown to the right (10 ng and 2 ng dsRNA control sample).

FIGS. 3A, 3B, 3C, and 3D shows (FIGS. 3A and 3B) the mRNA yield for in vitro transcription (IVT) including different concentrations of sodium perchlorate (lanes labeled according to Table 4), and (FIGS. 3C and 3D) two J2 mAb dot blots to detect dsRNA for the same IVT conditions as identified above. Each dot contains 1000 ng mRNA, and comparative dots are shown to the right (10 ng dsRNA control sample for FIG. 3C; 10 ng and 2 ng dsRNA control sample for FIG. 3D).

FIGS. 4A and 4B show (FIG. 4A) the mRNA yield for in vitro transcription (IVT) including different concentrations of thiourea (lanes labeled according to Table 5), and (FIG. 4B) a J2 mAb dot blot to detect dsRNA for the same IVT conditions as identified above. Each dot contains 1000 ng mRNA, and comparative dots are shown to the right (10 ng and 2 ng dsRNA control sample).

FIGS. 5A, 5B, and 5C show (FIGS. 5A and 5B) the mRNA yield for in vitro transcription (IVT) including different concentrations of sodium salicylate (lanes labeled according to Table 6), and (FIG. 5C) a J2 mAb dot blot to detect dsRNA for the same IVT conditions as identified above. Each dot contains 1000 ng mRNA, and comparative dots are shown to the right (10 ng and 2 ng dsRNA control sample).

FIGS. 6A, 6B, 6C, and 6D show (FIGS. 6A and 6B) the mRNA yield for in vitro transcription (IVT) including different concentrations of arginine (lanes labeled according to Table 7), and (FIGS. 6C and 6D) two J2 mAb dot blots to detect dsRNA for the same IVT conditions as identified above. Each dot contains 1000 ng mRNA, and comparative dots are shown (10 ng dsRNA control sample for FIG. 6C; 10 ng and 2 ng dsRNA control sample for FIG. 6D).

FIGS. 7A and 7B show (FIG. 7A) the mRNA yield for in vitro transcription (IVT) including different concentrations of ethanol (lanes labeled according to Table 8), and (FIG. 7B) a J2 mAb dot blot to detect dsRNA for the same IVT conditions as identified above. Each dot contains 1000 ng mRNA, and comparative dots are shown to the right (10 ng and 2 ng dsRNA control sample).

FIGS. 8A and 8B show (FIG. 8A) the mRNA yield for in vitro transcription (IVT) including different concentrations of 2-propanol (lanes labeled according to Table 9), and (FIG. 8B) a J2 mAb dot blot to detect dsRNA for the same IVT conditions as identified above. Each dot contains 1000 ng mRNA, and comparative dots are shown to the right (10 ng and 2 ng dsRNA control sample).

FIGS. 9A and 9B show (FIG. 9A) the mRNA yield for in vitro transcription (IVT) including different concentrations of n-butanol (lanes labeled according to Table 10), and (FIG. 9B) a J2 mAb dot blot to detect dsRNA for the same IVT conditions as identified above. Each dot contains 1000 ng mRNA, and comparative dots are shown to the right (10 ng and 2 ng dsRNA control sample).

FIGS. 10A, 10B, and 10C show (FIGS. 10A and 10B) the mRNA yield for in vitro transcription (IVT) including different concentrations of guanidine hydrochloride (lanes labeled according to Table 11), and (FIG. 10C) a J2 mAb dot blot to detect dsRNA for the same IVT conditions as identified above. Each dot contains 1000 ng mRNA, and comparative dots are shown to the right (10 ng and 2 ng dsRNA control sample).

FIG. 11 shows the mRNA yield for in vitro transcription (IVT) including different concentrations of SDS (lanes labeled according to Table 12).

FIGS. 12A and 12B show (FIG. 12A) the mRNA yield for in vitro transcription (IVT) including different concentrations of DMSO (lanes labeled according to Table 13), and (FIG. 12B) a J2 mAb dot blot to detect dsRNA for the same IVT conditions as identified above. Each dot contains 1000 ng mRNA, and comparative dots are shown to the right (10 ng and 2 ng dsRNA control sample).

FIGS. 13A and 13B show (FIG. 13A) the mRNA yield for in vitro transcription (IVT) including different concentrations of propylene glycol (lanes labeled according to Table 14), and (FIG. 13B) a J2 mAb dot blot to detect dsRNA for the same IVT conditions as identified above. Each dot contains 1000 ng mRNA, and comparative dots are shown to the right (10 ng and 2 ng dsRNA control sample).

FIGS. 14A and 14B show (FIG. 14A) the mRNA yield for in vitro transcription including different concentrations of urea and at different temperatures (lanes labeled according to Table 15). Lanes 1-5 correspond to IVT at 34° C. for 2 hrs, with 0, 0.4, 0.8, 1.2, and 1.6 M urea, respectively. Lanes 6-10 correspond to IVT at 37° C. for 2 hrs, with 0, 0.4, 0.8, 1.2, and 1.6 M urea, respectively. Lanes 11-15 correspond to IVT at 40° C. for 2 hrs, with 0, 0.4, 0.8, 1.2, and 1.6 M urea, respectively. FIG. 14B shows a J2 mAb dot blot to detect dsRNA for the same IVT conditions as identified above. Each dot contains 1000 ng mRNA, and comparative dots are shown to the right (10 ng and 2 ng dsRNA control sample). DS stands for drug substance, the same mRNA from in vitro transcription carried out in the absence of chaotropic agent(s), but purified via HPLC.

FIG. 15 shows a J2 mAb dot blot to detect dsRNA for the IVT conditions identified in Table 16, with the three left-hand dots corresponding to modified NTPs and the three righthand dots corresponding to regular NTP.

FIGS. 16A and 16B show two J2 mAb dot blots to detect dsRNA for the IVT conditions identified in Table 17, with FIG. 16A showing the 8 mL results and FIG. 16B showing the 600 mL results, and comparative dots are shown to the right (DS (drug substance) and 10 ng dsRNA control sample).

FIG. 17 shows a J2 mAb dot blot to detect dsRNA for the IVT conditions identified in Table 18, and comparative dots are shown to the right (DS (drug substance) and 10 ng dsRNA control sample).

FIG. 18 shows a J2 mAb dot blot to detect dsRNA for the IVT conditions identified in Table 19, and comparative dots are shown to the right (DS (drug substance) and 10 ng dsRNA control sample).

FIG. 19 shows a J2 mAb dot blot to detect dsRNA for the IVT conditions identified in Table 20, and comparative dots are shown to the right (DS (drug substance) and 10 ng dsRNA control sample).

FIGS. 20A and 20B show (FIG. 20A) a J2 mAb dot blot to detect dsRNA for IVT with vs. without formamide. Each dot contains 1000 ng mRNA, and a comparative dot is shown to the right (10 ng dsRNA control sample). FIG. 20B shows the mRNA yield for the IVT conditions identified above.

### DETAILED DESCRIPTION

It is to be understood that this disclosure is not limited to particular methods and experimental conditions described, as such methods and conditions may vary. It is also to be understood that the terminology used herein is for the purpose of describing particular embodiments only, and is not intended to be limiting, since the scope of the disclosure will be limited only by the appended claims.

Unless defined otherwise, all technical and scientific terms used herein have the same meaning as commonly understood by one of ordinary skill in the art to which this disclosure belongs. As used herein, the term “about,” when used in reference to a particular recited numerical value, means that the value may vary from the recited value by no more than 1%. For example, as used herein, the expression “about 100” includes 99 and 101 and all values in between (e.g., 99.1, 99.2, 99.3, 99.4, etc.). The terms “Figure” and “Fig.” are used interchangeably throughout the specification.

Although any methods and materials similar or equivalent to those described herein can be used in the practice or testing of the compositions and methods and uses, the preferred methods and materials are now described. All patents, applications and non-patent publications mentioned in this specification are incorporated herein by reference in their entireties.

### Definitions

The terms “reduce”, “minimize”, and “inhibit”, as used herein, or grammatical equivalents of the same, indicate values that are relative to a baseline measurement. For example, in the context of reducing, minimizing, or inhibiting dsRNA formation during the preparation of mRNA, an in vitro transcription reaction is carried out in the absence and in the presence of the tested chaotropic agent, and dsRNA quantities are subsequently compared for the same amount of mRNA produced from the two in vitro transcription reactions.

The terms “impurities” and “contaminants”, used interchangeably herein, refer to substances inside a confined amount of liquid, gas, or solid product, which differ from the chemical composition of the target material or compound, e.g., mRNA. Possible contaminants and impurities resulting during the synthesis of mRNA/in vitro transcription include, without limitation, polymerases, dsRNA, DNA, unincorporated NTPs, prematurely aborted RNA sequences, salts, and DTT.

As used herein, the term “in vitro” refers to events that occur in an artificial environment, e.g., in a test tube or reaction vessel, in cell culture, etc., instead of within a multi-cellular organism. In vitro synthesis, as used herein, refers to the cell-free synthesis of biological macromolecules, e.g., mRNA, in a reaction mix comprising biological extracts and/or defined reagents.

The phrase “starting reaction mixture”, as used herein, refers to the mixture in which the transcription reaction/preparation of RNA (for example, mRNA) takes place. The phrases “starting reaction mixture” and “in vitro transcription reaction mixture” and “transcription mixture” are used interchangeably herein. In certain embodiments, the chaotropic agent is added to the “starting reaction mixture” at the initiation of the reaction. In certain embodiments, the chaotropic agent is added to the “starting reaction mixture” after the transcription reaction has been initiated/carried out for a period of time.

The term “chaotropic agent”, as used herein, refers to a molecule that disrupts non-covalent bonds (for example, without limitation, hydrogen bonds, van der Waals forces, and hydrophobic interactions). Chaotropic agents can disrupt the structure of/denature macromolecules such as proteins and nucleic acids.

As used herein, the term “isolated” refers to a substance that has been (1) separated from at least some of the components with which it is associated when initially produced, whether in nature or in an experiment. The term “isolated” also refers to a substance that has been (2) produced, prepared, and/or manufactured by the hand of man. In certain embodiments, the RNA synthesized using the methods disclosed herein is isolated. In one embodiment, additional steps carried out to purify the RNA product remove enzymes, plasmids, and/or other small molecules to get the final pure “isolated” RNA (for example, isolated mRNA).

As used herein, the term “pure”, when used to describe a substance, indicates that the substance is substantially free of other components. The percent purity of isolated substances can be calculated and does not include excipients (e.g., buffer, solvent, water, etc.).

The term “substantially free”, as used herein, refers to a state in which relatively little or no amount of a substance to be removed (e.g., contaminants/impurities) are present. For example, “substantially free of dsRNA” means that dsRNA is present at a level less than about 2%, about 1.5%, about 1.0%, 0.9%, 0.8%, 0.7%, 0.6%, 0.5%, 0.4%, 0.3%, 0.2%, 0.1%, or less (w/w) of the contaminant/impurity.

As used herein, the term “nucleic acid” refers to any biomolecule/macromolecule that is or can be incorporated into a polynucleotide chain. In some embodiments, a nucleic acid is a compound and/or substance that is or can be incorporated into a polynucleotide chain via a phosphodiester linkage. In some embodiments, “nucleic acid” refers to individual nucleic acid residues (e.g., nucleotides and/or nucleosides). In some embodiments, “nucleic acid” refers to a polynucleotide chain comprising individual nucleic acid residues. As used herein, the term “nucleic acid” may encompass RNA (for example, mRNA, tRNA, rRNA, miRNA) or DNA. As used herein, the term “nucleic acid” includes nucleic acid analogs, for example, analogs having other than a phosphodiester backbone. For chemically synthesized molecules, nucleic acids can comprise nucleoside analogs such as analogs having chemically modified bases or sugars, backbone modifications, etc.

As used herein, the term “poly A tail” refers to a chain of adenine nucleotides. In certain embodiments, the poly A tail is to be added to an RNA transcript. In certain embodiments, the poly A tail exists at the 3′ end of an RNA transcript. A poly A tail is typically about 5 to about 300 nucleotides in length.

The term “DNA template”, as used herein, refers to a polynucleotide template for RNA polymerase (for example, for in vitro transcription). The DNA template comprises a sequence for a gene of interest.

As used herein, the term “subject” refers to a human or any non-human animal (e.g., mouse, rat, rabbit, dog, cat, cattle, swine, sheep, horse, or primate). The terms “subject”, “individual”, and “patient” are used interchangeably herein.

Additional definitions for the following terms and other terms are set forth throughout the specification.

Messenger Ribonucleic Acid (mRNA)

The term “messenger RNA (mRNA)”, as used herein, refers to a polynucleotide that encodes at least one polypeptide. The terms “mRNA” and “RNA transcript” are used interchangeably herein. An “RNA transcript” refers to a ribonucleic acid produced by an in vitro transcription reaction using a DNA template and an RNA polymerase. An RNA transcript typically includes the coding sequence for a gene of interest (i.e., a polynucleotide encoding a polypeptide or protein of interest) and, in certain embodiments, a poly A tail. Polypeptides and/or proteins of interest may be selected from, without limitation, biologics, antibodies, vaccines, therapeutic proteins or peptides, cell penetrating peptides, secreted proteins, plasma membrane proteins, cytoplasmic or cytoskeletal proteins, intracellular membrane bound proteins, nuclear proteins, proteins associated with human disease, targeting moieties, and any proteins having diagnostic or therapeutic utility. mRNA as described herein encompasses modified and unmodified RNA. mRNA as described herein may contain one or more coding and non-coding regions.

mRNA integrity is assessed to determine whether or not the mRNA is intact (vs. degraded). mRNA integrity can be determined using methods well known in the art, for example, RNA agarose gel electrophoresis (Ausubel, et al. 1997 Current Protocols in Molecular Biology), capillary electrophoresis, HPLC, and size exclusion chromatography. RNA integrity (for example, mRNA integrity) is distinct from RNA purity.

mRNAs may be synthesized according to any of a variety of known methods. For example, mRNAs may be synthesized via in vitro transcription (IVT). In vitro transcription allows for template-directed synthesis of RNA molecules of any sequence from short oligonucleotides to oligonucleotides of several kilobases in μg to kg quantities. Briefly, IVT is typically carried out employing a linear or circular DNA template containing a promoter, a pool of ribonucleotide triphosphates, a buffer system that may include DTT and magnesium ions, and an appropriate RNA polymerase (e.g., T3, T7 or SP6 RNA polymerase), pyrophosphatase, and/or RNAse inhibitor.

According to certain embodiments, the methods described herein are used to prepare RNA (for example, mRNA) of various lengths. In some embodiments, the synthesized mRNA is of or greater than about 200 base pairs (bp) to about 20 kb, i.e., about 200 bp, 300 bp, 400 bp, 500 bp, 600 bp, 700 bp, 800 bp, 900 bp, 1 kb, 1.5 kb, 2 kb, 2.5 kb, 3 kb, 3.5 kb, 4 kb, 4.5 kb, 5 kb 6 kb, 7 kb, 8 kb, 9 kb, 10 kb, 11 kb, 12 kb, 13 kb, 14 kb, 15 kb, or 20 kb in length. In certain embodiments, the synthesized mRNAs are about 1 kb to about 5 kb in length.

In certain embodiments, the synthesized mRNA includes 5′ and/or 3′ untranslated region(s).

In certain embodiments, a 5′ cap and/or a 3′ poly A tail can be added independently to the RNA product (for example, transcript product of in vitro transcription (IVT)). In other embodiments, a 5′ cap can be added during IVT. In still other embodiments, a 3′ poly A tail is encoded into the DNA template (reagent of the transcription reaction). Thus, the methods described herein can, in specific embodiments, include adding a 5′ cap and/or a 3′ poly A tail during IVT.

The methods described herein are scalable and allow cost-effective, large-scale production of pharmaceutical-grade mRNA. The mRNA yield (e.g., from in vitro transcription) can be quantified, for example, via UV absorbance, RNA blotting or Northern blotting, ribonuclease protection assays, Q-PCR (e.g., QRT-PCR). In terms of the scalability of the RNA (for example, mRNA) prepared according to the methods disclosed herein, it can be prepared on the μg, mg, g, and even kg scale. In certain embodiments of the methods disclosed herein, about 1 to about 7 mg/mL, about 1 to about 6 mg/mL, about 1 to about 5 mg/mL, about 1 to about 4 mg/mL, about 1.5 to about 3.5 mg/mL mRNA is synthesized.

In certain embodiments, mRNA prepared using a method described herein maintain a high degree of integrity. As used herein, the term “mRNA integrity” generally refers to the quality of the mRNA after transcription. mRNA integrity can be determined using methods well known in the art, for example, by RNA agarose gel electrophoresis (e.g., Ausubel, et al. 1997 Current Protocols in Molecular Biology), capillary electrophoresis, HPLC, size exclusion chromatography. In further embodiments, mRNA synthesized according to the methods of the instant disclosure has a comparable integrity to mRNA synthesized without the use of at least one chaotropic agent. In still further embodiments, mRNA synthesized according to the methods of the instant disclosure has an integrity no more than about 15% less than the integrity of mRNA synthesized without the use of at least one chaotropic agent. In yet further embodiments, mRNA synthesized according to the methods of the instant disclosure has an integrity no more than about 10% less, about 9% less, about 8% less, about 7% less, about 6% less, about 5% less, about 4% less, about 3% less, about 2% less, or about 1% less than the integrity of mRNA synthesized without the use of at least one chaotropic agent.

Generally speaking, the integrity of mRNA depends on transcription conditions, DNA template length and quality, and even different evaluation methods.

In certain embodiments, the purity of the mRNA is determined by evaluating the presence or absence of contaminants. For example, the presence or absence of dsRNA can be determined using denaturing gel electrophoresis, native gel electrophoresis, anti-dsRNA antibody, intact mass spectrometry, and/or controls for dsRNA.

### Transcription Reaction Mixture

The in vitro transcription mixture (starting reaction mixture) generally comprises a DNA template, a transcription buffer, nucleotide triphosphates (NTPs) (natural or modified), magnesium ion, and RNA polymerase(s). RNase inhibitor(s) and/or pyrophosphatase can be added to an in vitro transcription mixture in certain embodiments.

RNA polymerases are generally known in the art. Exemplary RNA polymerases include, without limitation, phage RNA polymerase (for example, T7 RNA polymerase, T3 RNA polymerase, SP6 RNA polymerase) and mutant polymerases (for example, incorporating modified nucleic acids).

RNA polymerase in the transcription mixture is, in certain embodiments, at a final concentration of about 1000 to about 50,000 U/mL, in further embodiments, at a final concentration of about 1000 to about 12,000 U/mL. DNA template in the transcription mixture is, in certain embodiments, at a final concentration of about 1 to about 200 nM, in further embodiments, at a final concentration of about 5 to about 70 nM. Nucleotide triphosphates (NTPs) in the transcription mixture are, in certain embodiments, at a final concentration of about 0.5 to about 10 mM. Magnesium/magnesium ions and DTT (or some other reducing agent) are often included in transcription buffers. Magnesium in the transcription mixture is, in certain embodiments, at a final concentration of about 12 to about 60 mM. Buffer (for example, HEPES or Tris at a pH of about 7 to about 8.5) in the transcription mixture is, in certain embodiments, at a final concentration of about 10 to about 250 mM. Spermidine (or some other polyamine compound) in the transcription mixture is, in certain embodiments, at a final concentration of about 0.1 to about 5 mM. T7 RNA polymerase in the transcription mixture is, in certain embodiments, at a final concentration of about 1000 to about 10,000 U/mL. RNase inhibitor in the transcription mixture is, in certain embodiments, at a final concentration of about 500 to about 2000 U/mL. Pyrophosphatase in the transcription mixture is, in certain embodiments, at a final concentration of about 0.1 to about 10 U/mL.

The “starting reaction mixture” is the mixture in which the transcription reaction/preparation of RNA (for example, mRNA) takes place. The starting reaction mixture comprises, in certain embodiments, 50 ng/μL DNA plasmid template, rNTPs (5 mM), CleanCap AG (4 mM), reaction buffer (10× buffer: 400 mM HEPES (pH 7.2-7.5), 100 mM DTT, 20 mM spermidine, 0.02% triton X-100, 165 mM Mg2+), T7 RNA polymerase (about 4000 U/mL), RNase inhibitor (about 1000 U/mL), and inorganic pyrophosphatase (about 2 U/mL). In further embodiments, the starting reaction mixture is prepared in Rnase-free water. In still further embodiments, a chaotropic agent is added to the starting reaction mixture to get a final volume of about 40 μL.

### Chaotropic Agents

Chaotropic agents generally create a denaturing environment for nucleic acids and proteins. Thus, it is contemplated herein to use chaotropic agents to reduce the nucleic acid interactions that cause the formation of dsRNA during in vitro transcription (IVT). Chaotropic agents contemplated for use in the methods, uses, and compositions of the disclosure include, without limitation, urea, formamide, sodium salicylate, ethanol, sodium perchlorate, arginine, n-butanol, thiourea, and 2-propanol. Additional chaotropic agents include, without limitation, guanidinium chloride/guanidine hydrochloride, guanidine thiocyanate, lithium perchlorate, lithium acetate, propylene glycol, phenol, and DMSO.

In specific embodiments of the invention, chaotropic agents are used at, without limitation, any of the concentrations: formamide (0.4 M, 0.8 M, 1.2 M, 1.6 M, 2.0 M, 2.4 M, or 2.8 M); sodium perchlorate (0.01 M, 0.025 M, 0.050 M, 0.075 M, 0.2 M, 0.8 M, or 1.6 M); sodium salicylate (0.005 M, 0.010 M, 0.020 M, 0.040 M, 0.1 M, 0.2 M, 0.3 M, or 0.4 M), arginine (0.010 M, 0.025 M, 0.050 M, 0.075 M, 0.2 M, 0.4 M, or 0.8 M); ethanol (0.4 M, 0.8 M, 1.2 M, or 1.6 M); thiourea (0.1 M, 0.2 M, 0.4 M, or 0.8 M); 2-propanol (0.4 M, 0.8 M, 1.2 M, or 1.6 M); n-butanol (0.08 M, 0.16 M, 0.24 M, or 0.32 M); guanidine hydrochloride (0.005 M, 0.010 M, 0.020 M, 0.040 M, 0.4 M, 0.8 M, 1.2 M, or 1.6 M); sodium dodecyl sulfate (SDS) (0.001735 M, 0.008675 M, 0.0347 M, or 0.1388 M); dimethyl sulfoxide (DMSO) (0.4 M, 0.8 M, 1.2 M, or 1.6 M); or propylene glycol (0.4 M, 0.8 M, 1.2 M, or 1.6 M).

The chaotropic agents evaluated herein include:

TABLE 1
list of chaotropic agents tested

Agent
Urea
Formamide
Guanidine HCl
Sodium Salicylate
DMSO
Ethanol
SDS
Sodium Perchlorate
Propylene Glycol
Arginine
n-butanol
Thiourea
2-propanol

### Compositions

The disclosure provides compositions comprising mRNA prepared according to the method of any one of the preceding claims, wherein the composition is substantially free of dsRNA. These compositions can be used for therapeutic purposes. In certain embodiments, these compositions can be further formulated for administration to a subject.

### EXAMPLES

The following examples are put forth so as to provide those of ordinary skill in the art with a complete disclosure and description of how to make and use the methods and compositions of the disclosure, and are not intended to limit the scope of what the inventors regard as their disclosure. Efforts have been made to ensure accuracy with respect to numbers used (e.g., amounts, temperature, etc.) but some experimental errors and deviations should be accounted for. Unless indicated otherwise, parts are parts by weight, molecular weight is average molecular weight, temperature is in degrees Centigrade, and pressure is at or near atmospheric.

### General Methods

Unless otherwise specified, standard in vitro transcription (IVT) was used in the examples. Standard IVT runs were prepared as a stock solution and aliquoted to mix with different chaotropic agents at various concentrations immediately or within a certain time frame. The IVT in the presence of chaotropic agents were usually kept for 2-3 hours at selected temperature, i.e., around 37° C. After DNase I was added to digest DNA plasmid, the mRNA samples were purified by spin column. Yield, dsRNA content, and mRNA integrity were assessed for the impact of the selected chaotropic agents at the tested concentration. dsRNA content was assessed by J2 anti-dsRNA mAb dot blot. HPLC-purified mRNA Drug Substance and dsRNA control sample were used to quantify dsRNA content. The results from different membrane should not be compared directly without referring to dsRNA control. Yield was evaluated by Nanodrop (UV/Vis) following manufacturer's instruction, and mRNA integrity was measured by Fragment Analyzer followed by manufacturer's instruction. 0.7% agarose gel was used to confirm that correct mRNA was produced and evaluate the yield visually.

### In Vitro Transcription Recipe

The standard IVT recipe consisted of: 5 mM of each NTP, 50 ng/μL plasmid, 16.5 mM Mg2+, 40 mM HEPES, 10 mM DTT, 0.002% Triton X-100, RNase inhibitor (1 kU/mL), E. Coli Inorganic Pyrophosphastase (2 U/mL), and T7 RNA polymerase (4 kU/mL). CleanCap AG (4 mM) was used if a co-transcriptional manner was applied. Alternatively, Vaccinia Capping Enzyme (VCE) and 2′-O-methyltransferase were used for post-transcriptional capping. Whether modified NTP or not, whether CleanCap AG or VCE, and plasmid information are listed in each specific example, below.

### J2 mAb Dot Blot

J2 mAb dot blot protocol: the same amount of purified mRNA was applied on each sample dot for a fair comparison, and the estimated dsRNA concentration was calculated based on 10 ng dsRNA control on the same membrane. More specifically, 1000 ng of each mRNA sample and 10 ng/2 ng dsRNA control sample were used for J2 mAb dot blot, unless otherwise specified. Drug Substance (DS) refers to HPLC-purified mRNA drug substance. After blocking the membrane with 3.3% non-fat milk in 1×TBST buffer for 1 h, J2 antibody was applied in 1×TBST buffer to incubate the membrane for 1 h. After washing 6 times, anti-mouse IgG-HRP antibody was applied to the membrane for 1 h. After washing 6 times, chemiluminescence was developed to show the intensity of the dsRNA.

### Gel Electrophoresis

Gel electrophoresis protocol: the same volume (10 μL of 40-fold diluted IVT samples post-DNase I treatment) of mRNA samples prior to spin column purification were used, and 6000-nt RiboRuler RNA ladder was used to show the length of correct mRNA. 10000× diluted GelRed was used in a pre-stained manner to signal the bands. Gel was run at 100V for 1 h at room temperature and imaged by Amersham Imager 680.

For screening, 20-200 μL IVT was usually used for the convenience of spin column purification. Larger volume as specified was used to further demonstrate the broad application of chaotropic agents to reduce formation of dsRNA during IVT.

### Example 1. Screening Chaotropic Agent Urea for dsRNA Reduction

Urea was tested for the purpose of dsRNA reduction during in vitro transcription (IVT). Glycogen debranching enzyme-encoding messenger RNA (mRNA) was synthesized by in vitro transcription of a linearized DNA plasmid template. 5′ capping structure was included through capping agent in a co-transcriptional manner, and 3′ polyA tail was encoded in the DNA plasmid. A mixture of 50 ng/μL DNA plasmid template, rNTPs (5 mM each, N1-methylpseudouridine (N1mΨ) used instead of UTP), CleanCap AG (4 mM), urea (X M), reaction buffer (10× buffer: 400 mM HEPES (pH 7.2-7.5), 100 mM DTT, 20 mM spermidine, 0.02% triton X-100, 165 mM Mg²⁺), T7 RNA polymerase (4000 U/mL), Rnase inhibitor (1000 U/mL), inorganic pyrophosphatase (2 U/mL) was prepared in Rnase-free water to get a final volume of 40 μL.

Urea concentrations from 0 M to 1.6 M were evaluated. The dot blot results indicated that increasing urea concentration effectively reduced dsRNA during IVT. mRNA integrity was slightly increased or comparable.

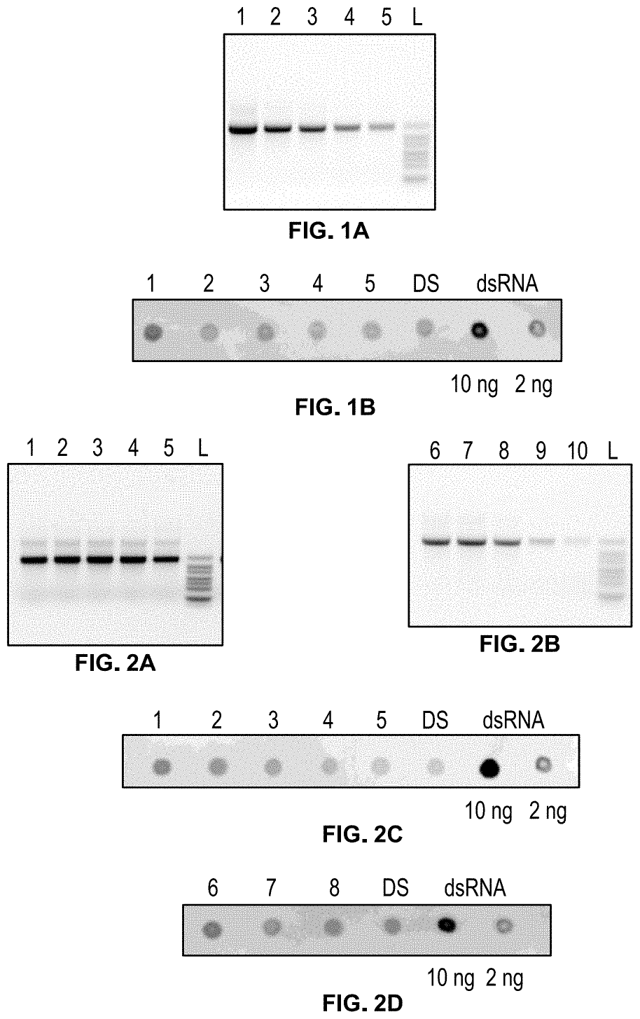

The results of IVT in the presence of different concentrations of urea are shown in Table 2, below. The impact of urea on mRNA yield and dsRNA is shown in FIG. 1.

TABLE 2
Results of IVT in the presence of urea at different concentrations

| | Urea | | Yield | dsRNA | | dsRNA in |
|---|---|---|---|---|---|---|
| Samples | concentration | | (mg/mL) | (ppt) | Integrity | DS (ppt) |
| Earlier | 0 | M | 2.824 | 6.43 | N. D. | Unable to quantify |
| samples | 0.1 | M | 2.923 | 5.25 | N. D. | dsRNA due to dark |
| | 0.2 | M | 2.930 | 4.69 | N. D. | background, but dsRNA |
| | 0.4 | M | 2.825 | 1.53 | N. D. | lower than that |
| | 0.8 | M | 2.458 | 1.44 | N. D. | of 0.8 M urea |
| | 0.4 | M | 2.830 | 4.05 | N. D. | Unable to quantify |
| | 0.8 | M | 2.763 | 2.40 | N. D. | dsRNA due to dark |
| | 1.2 | M | 2.525 | 1.23 | N. D. | background, but dsRNA |
| | 1.6 | M | N. D. | N. D. | N. D. | higher than that of 1.2M |
| | | | | | | urea |
| 1 | 0 | M | 3.633 | 5.71 | 75.9% | 3.14 |
| 2 | 1.2 | M | 2.647 | 2.44 | 77.1% | |
| 3 | 1.3 | M | 2.179 | 3.26 | 76.5% | |
| 4 | 1.4 | M | 1.542 | 2.20 | 75.8% | |
| 5 | 1.5 | M | 1.012 | 2.80 | 76.2% | |

### Example 2. Screening Chaotropic Agent Formamide for dsRNA Formation Reduction

Given the benefits seen with the addition of the chaotropic agent urea, in vitro transcription of glycogen debranching enzyme-encoding mRNA was used as an example for the screening of the impact of additional chaotropic agents on the dsRNA reduction. A mixture of 50 ng/μL linearized DNA plasmid template, rNTPs (5 mM each, N1-methylpseduouridine used instead of UTP), CleanCap AG (4 mM), formamide (X M), reaction buffer (10× buffer: 400 mM HEPES (pH 7.2-7.5), 100 mM DTT, 20 mM spermidine, 0.02% triton X-100, 165 mM Mg2+), T7 RNA polymerase (4000 U/mL), Rnase inhibitor (1000 U/mL), inorganic pyrophosphatase (2 U/mL) was prepared in RNase-free water to get a final volume of 40 μL. The transcription mixture was incubated at about 37° C. for 2 h and 20 minutes, followed by DNase I treatment for 15 minutes. Synthesized mRNA was purified by silica membrane-based spin columns.

Formamide was tested for the purpose of dsRNA reduction during IVT. Formamide concentrations from 0 M to 2.8 M were evaluated. Formamide also effectively reduced dsRNA during IVT. mRNA integrity slightly increased or remained comparable.

The results of IVT in the presence of different concentrations of formamide are shown in Table 3, below. The impact of formamide on mRNA yield and dsRNA is shown in FIG. 2.

TABLE 3
Results of IVT in the presence of formamide

| | Formamide | | Yield | dsRNA | | dsRNA in |
|---|---|---|---|---|---|---|
| samples | Concentration | | (mg/mL) | (ppt) | Integrity | DS (ppt) |
| 1 | 0 | M | 3.762 | 5.95 | 75.2% | 2.04 |
| 2 | 0.4 | M | 4.037 | 5.12 | 74.0% | |
| 3 | 0.8 | M | 3.519 | 3.57 | 74.8% | |
| 4 | 1.2 | M | 3.860 | 2.66 | 75.7% | |
| 5 | 1.6 | M | 3.457 | 2.38 | 77.4% | |
| 6 | 0 | M | 3.633 | 5.71 | 75.9% | 3.82 |
| 7 | 1.6 | M | 3.435 | 3.63 | 75.6% | |
| 8 | 2.0 | M | 2.427 | 4.06 | 76.4% | |
| 9 | 2.4 | M | N. D. | N. D. | N. D. | |
| 10 | 2.8 | M | N. D. | N. D. | N. D. | |

### Example 3. Screening Chaotropic Agent Sodium Perchlorate for dsRNA Formation Reduction

Because of perchlorate's known chaotropic action, sodium perchlorate was tested for the purpose of dsRNA reduction during IVT. Sodium perchlorate concentrations from 0 M to 1.6 M were evaluated. Sodium perchlorate at low concentration effectively reduced dsRNA during IVT. mRNA integrity slightly increased.

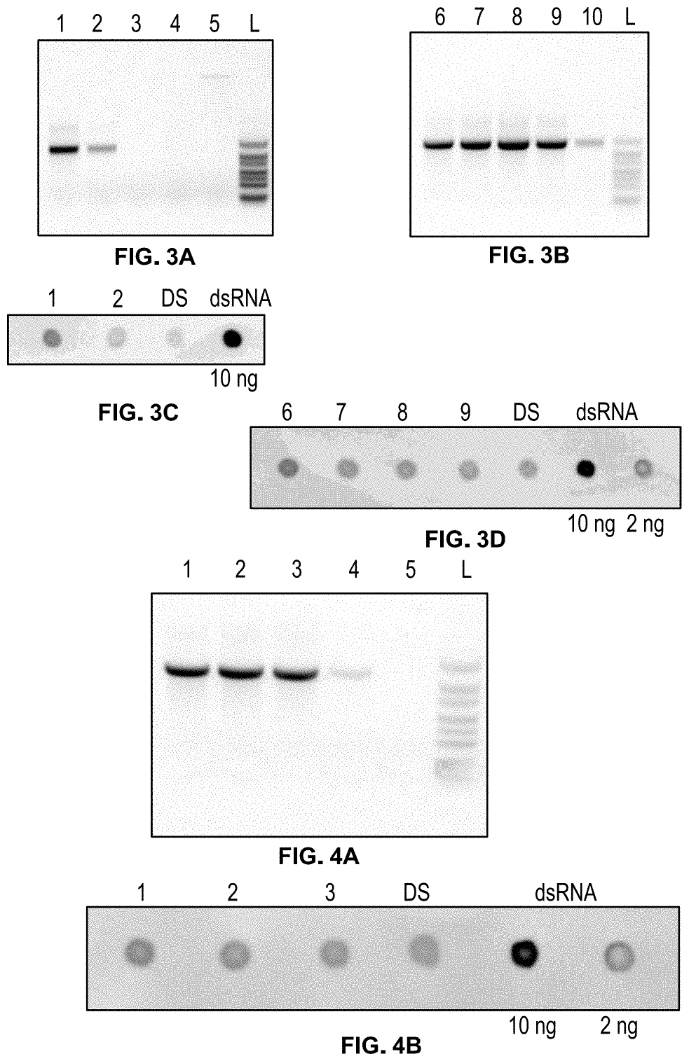

The same IVT composition used in Example 1, except chaotropic agent, was applied in the instant example. The results of IVT in the presence of different concentrations of sodium perchlorate are shown in Table 4, below. The impact of sodium perchlorate on mRNA yield and dsRNA is shown in FIG. 3.

TABLE 4
Results of IVT in the presence of sodium perchlorate

| | NaClO₄ | | Yield | dsRNA | | dsRNA in |
|---|---|---|---|---|---|---|
| Sample | Concentration | | (mg/mL) | (ppt) | Integrity | DS (ppt) |
| 1 | 0 | M | 3.579 | 4.25 | 74.6% | 0.91 |
| 2 | 0.05 | M | 1.145 | 2.00 | 78.5% | |
| 3 | 0.2 | M | N. D. | N. D. | N. D. | |
| 4 | 0.8 | M | N. D. | N. D. | N. D. | |
| 5 | 1.6 | M | N. D. | N. D. | N. D. | |
| 6 | 0 | M | 3.633 | 5.71 | 75.9% | 2.81 |
| 7 | 10 | mM | 4.204 | 3.72 | 77.4% | |
| 8 | 25 | mM | 4.202 | 3.83 | 76.3% | |
| 9 | 50 | mM | 3.082 | 3.34 | 77.5% | |
| 10 | 75 | mM | N. D. | N. D. | N. D. | |

### Example 4. Screening Chaotropic Agent Thiourea for dsRNA Formation Reduction

Thiourea was tested for the purpose of dsRNA reduction during IVT. Thiourea concentrations from 0 M to 0.8 M were evaluated. Thiourea at low concentration moderately reduced dsRNA during IVT. At high concentrations, thiourea shut down IVT.

The same IVT composition used in Example 1, except chaotropic agent, was applied in the instant example. The results of IVT in the presence of different concentrations of thiourea are shown in Table 5, below. The impact of thiourea on mRNA yield and dsRNA is shown in FIG. 4.

TABLE 5
Results of IVT in the presence of thiourea

| | | | | dsRNA | | |
|---|---|---|---|---|---|---|
| | Thiourea | | Yield | (ng/ug of | | dsRNA |
| Sample | concentration | | (mg/mL) | mRNA) | purity | in DS |
| 1 | 0 | M | 3.443 | 4.95 | 71.6% | 3.75 |
| 2 | 0.1 | M | 3.829 | 4.43 | 74.3% | |
| 3 | 0.2 | M | 3.351 | 3.73 | 75.8% | |
| 4 | 0.4 | M | N. D. | N. D. | N. D. | |
| 5 | 0.8 | M | N. D. | N. D. | N. D. | |

### Example 5. Screening Chaotropic Agent Sodium Salicylate for dsRNA Formation Reduction

Sodium salicylate was tested for the purpose of dsRNA reduction during IVT. Sodium salicylate concentrations from 0 M to 0.4 M were evaluated. Sodium salicylate at low concentration moderately reduced dsRNA during IVT. At high concentrations, IVT was aborted.

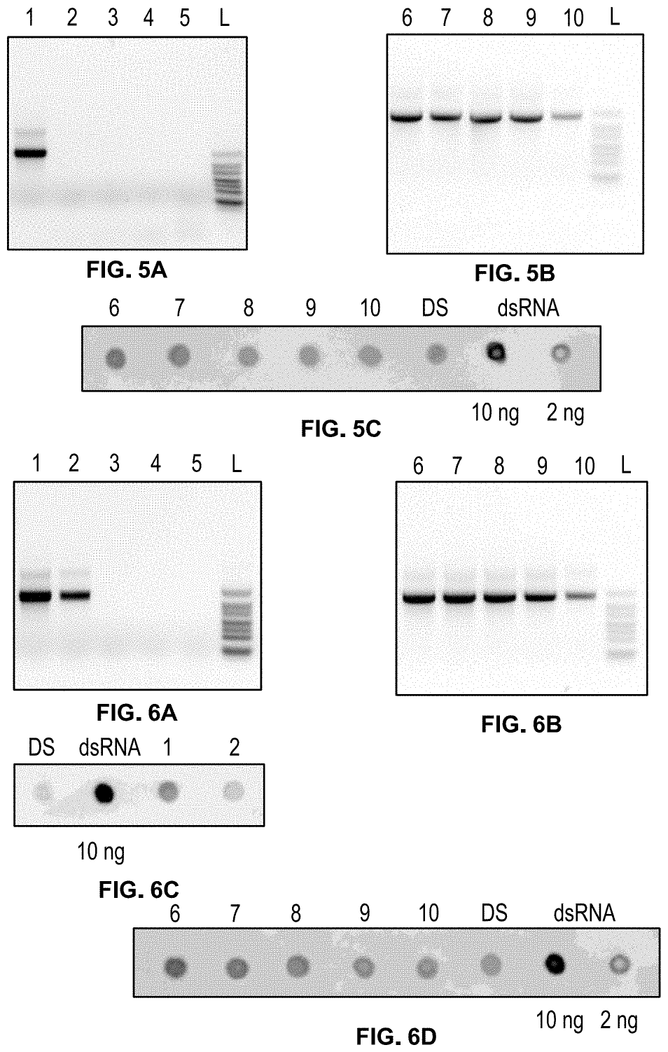

The same IVT composition used in Example 1, except chaotropic agent, was applied in the instant example. The results of IVT in the presence of different concentrations of sodium salicylate are shown in Table 6, below. The impact of sodium salicylate on mRNA yield and dsRNA is shown in FIG. 5.

TABLE 6
Results of IVT in the presence of sodium salicylate

| | Sodium | | yield | dsRNA | | dsRNA in |
|---|---|---|---|---|---|---|
| samples | salicylate Conc. | | (mg/mL) | (ppt) | Integrity | DS (ppt) |
| 1 | 0 | M | 3.762 | 5.95 | 75.2% | 2.04 |
| 2 | 0.1 | M | N. D. | N. D. | N. D. | |
| 3 | 0.2 | M | N. D. | N. D. | N. D. | |
| 4 | 0.3 | M | N. D. | N. D. | N. D. | |
| 5 | 0.4 | M | N. D. | N. D. | N. D. | |
| 6 | 0 | M | 3.633 | 5.71 | 75.9% | 3.31 |
| 7 | 5 | mM | 3.933 | 5.33 | 77.0% | |
| 8 | 10 | mM | 4.149 | 3.98 | 77.6% | |
| 9 | 20 | mM | 4.017 | 4.16 | 76.8% | |
| 10 | 40 | mM | 1.732 | 4.08 | 78.0% | |

### Example 6. Screening Chaotropic Agent Arginine for dsRNA Formation Reduction

Arginine was tested for the purpose of dsRNA reduction during IVT. Arginine concentrations from 0 M to 0.8 M were evaluated. Arginine at low concentration moderately reduced dsRNA during IVT. At high concentrations, IVT was shut down.

The same IVT composition used in Example 1, except chaotropic agent, was applied in the instant example. The results of IVT in the presence of different concentrations of arginine are shown in Table 7, below. The impact of arginine on mRNA yield and dsRNA is shown in FIG. 6.

TABLE 7
Results of IVT in the presence of Arginine

| | | yield | dsRNA | | dsRNA in |
|---|---|---|---|---|---|
| samples | Arginine Conc. | (mg/mL) | (ppt) | Integrity | DS (ppt) |
| 1 | Blank | 3.579 | 4.34 | 74.6% | 0.88 |
| 2 | 0.05 M arginine | 1.391 | 1.62 | 78.0% | |
| 3 | 0.2 M arginine | N. D. | N. D. | N. D. | |
| 4 | 0.4 M arginine | N. D. | N. D. | N. D. | |
| 5 | 0.8 M arginine | N. D. | N. D. | N. D. | |
| 6 | Blank | 3.633 | 5.71 | 75.9% | 3.12 |
| 7 | 10 mM arginine | 3.957 | 5.84 | 75.2% | |
| 8 | 25 mM arginine | 3.387 | 5.19 | 74.1% | |
| 9 | 50 mM arginine | 3.212 | 4.11 | 75.0% | |
| 10 | 75 mM arginine | 1.740 | 4.15 | 78.3% | |

### Example 7. Screening Chaotropic Agent Ethanol for dsRNA Formation Reduction

Ethanol was tested for the purpose of dsRNA reduction during IVT. Ethanol concentrations from 0 M to 1.6 M were evaluated. Ethanol at high concentration moderately reduced dsRNA during IVT.

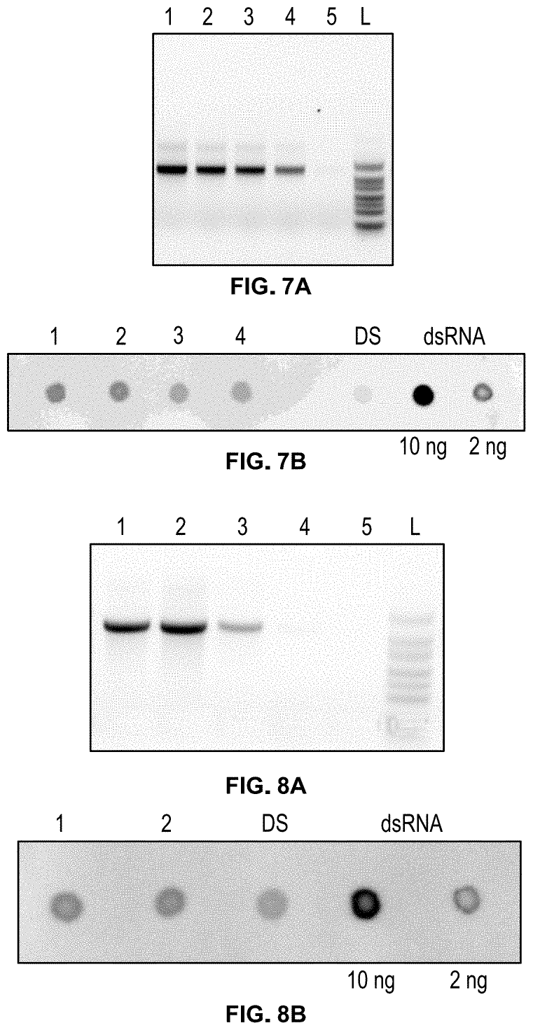

The same IVT composition used in Example 1, except chaotropic agent, was applied in the instant example. The results of IVT in the presence of different concentrations of arginine are shown in Table 8, below. The impact of arginine on mRNA yield and dsRNA is shown in FIG. 7.

TABLE 8
Results of IVT in the presence of ethanol

| | | | | | dsRNA |
|---|---|---|---|---|---|
| | Ethanol | Yield | dsRNA | | in DS |
| Sample | Concentration | (mg/mL) | (ppt) | Integrity | (ppt) |
| 1 | 0 M | 3.579 | 6.00 | 74.6% | 0.81 |
| 2 | 0.4 M | 2.256 | 5.70 | 77.6% | |
| 3 | 0.8 M | 2.052 | 4.21 | 74.2% | |
| 4 | 1.2 M | 1.282 | 3.59 | 78.3% | |
| 5 | 1.6 M | N. D. | N. D. | N. D. | |

### Example 8. Screening Chaotropic Agent 2-Propanol for dsRNA Formation Reduction

2-propanol was tested for the purpose of dsRNA reduction during IVT. 2-propanol concentration from 0 M to 1.6 M was evaluated. 2-propanol at low concentration moderately reduced dsRNA during IVT. At high concentrations, 2-propanol aborted IVT.

The same IVT composition used in Example 1, except chaotropic agent, was applied in the instant example. The results of IVT in the presence of different concentrations of 2-propanol are shown in Table 9, below. The impact of 2-propanol on mRNA yield and dsRNA is shown in FIG. 8.

TABLE 9
Results of IVT in the presence of 2-propanol

| | | | | | dsRNA |
|---|---|---|---|---|---|
| | 2-propanol | Yield | dsRNA | | in DS |
| Sample | concentration | (mg/mL) | (ppt) | Integrity | (ppt) |
| 1 | 0 M | 3.443 | 4.54 | 71.6% | 3.18 |
| 2 | 0.4 M | 3.537 | 4.01 | 67.4% | |
| 3 | 0.8 M | N. D. | N. D. | N. D. | |
| 4 | 1.2 M | N. D. | N. D. | N. D. | |
| 5 | 1.6 M | N. D. | N. D. | N. D. | |

### Example 9. Screening Chaotropic Agent n-Butanol for dsRNA Formation Reduction

n-butanol was tested for the purpose of dsRNA reduction during IVT. n-butanol concentrations from 0 M to 0.32 M were evaluated. n-butanol at low concentration moderately reduced dsRNA during IVT.

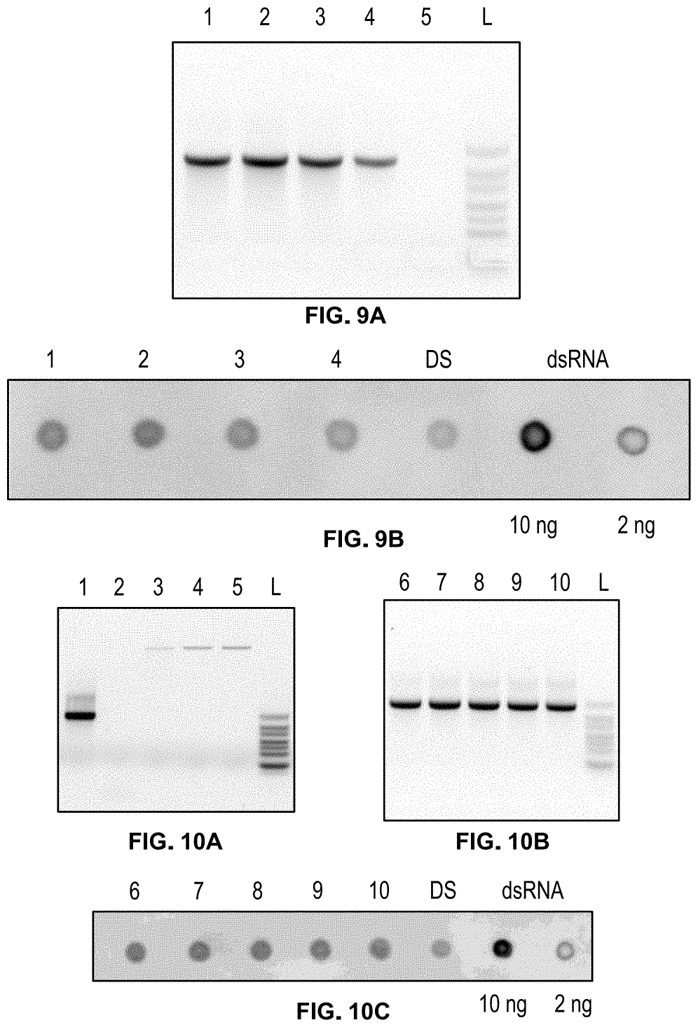

The same IVT composition used in Example 1, except chaotropic agent, was applied in the instant example. The results of IVT in the presence of different concentrations of n-butanol are shown in Table 10, below. The impact of n-butanol on mRNA yield and dsRNA is shown in FIG. 9.

TABLE 10
Results of IVT in the presence of n-butanol

| | | | | | dsRNA |
|---|---|---|---|---|---|
| | n-butanol | Yield | dsRNA | | in DS |
| Sample | concentration | (mg/mL) | (ppt) | Integrity | (ppt) |
| 1 | 0 M | 3.443 | 4.41 | 71.6% | 2.2 |
| 2 | 0.08 M | 3.821 | 4.82 | 72.4% | |
| 3 | 0.16 M | 3.230 | 4.05 | 73.9% | |
| 4 | 0.24 M | 1.644 | 3.43 | 75.4% | |
| 5 | 0.32 M | N. D. | N. D. | N. D. | |

### Example 10. Screening Chaotropic Agent Guanidine Hydrochloride for dsRNA Formation Reduction

Guanidine hydrochloride was tested for the purpose of dsRNA reduction during IVT. Guanidine hydrochloride concentrations from 0 M to 1.6 M were evaluated. Guanidine hydrochloride at low concentration did not reduce dsRNA during IVT. At high concentrations, Guanidine hydrochloride shut down IVT.

The same IVT composition used in Example 1, except chaotropic agent, was applied in the instant example. The results of IVT in the presence of different concentrations of guanidine hydrochloride are shown in Table 11, below. The impact of guanidine hydrochloride on mRNA yield and dsRNA is shown in FIG. 10.

TABLE 11
Results of IVT in the presence of guanidine hydrochloride

| | | | | | | dsRNA |
|---|---|---|---|---|---|---|
| | | | Yield | dsRNA | | in DS |
| Samples | Guanidine Conc | | (mg/mL) | (ppt) | Integrity | (ppt) |
| 1 | 0 | M | 3.762 | 5.95 | 75.2% | 2.04 |
| 2 | 0.4 | M | N. D. | N. D. | N. D. | |
| 3 | 0.8 | M | N. D. | N. D. | N. D. | |
| 4 | 1.2 | M | N. D. | N. D. | N. D. | |
| 5 | 1.6 | M | N. D. | N. D. | N. D. | |
| 6 | 0 | M | 3.633 | 4.92 | 75.9% | 3.03 |
| 7 | 5 | mM | 3.945 | 5.75 | 72.7% | |
| 8 | 10 | mM | 3.981 | 5.62 | 75.3% | |
| 9 | 20 | mM | 4.085 | 5.08 | 76.7% | |
| 10 | 40 | mM | 3.712 | 5.22 | 76.6% | |

### Example 11. Screening Chaotropic Agent SDS for dsRNA Formation Reduction

SDS was tested for the purpose of dsRNA reduction during IVT. SDS concentrations from 0.05% (1.735 mM) to 4% (138.8 mM) were evaluated. SDS shut down IVT completely at all tested concentrations.

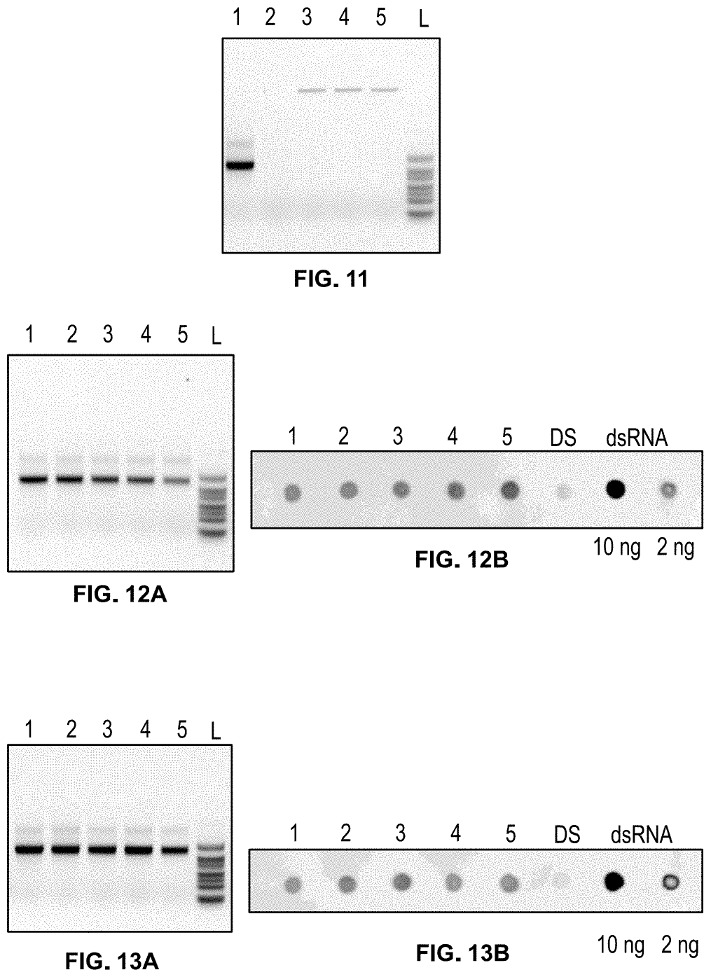

The same IVT composition used in Example 1, except chaotropic agent, was applied in the instant example. The results of IVT in the presence of different concentrations of SDS are shown in Table 12, below. The impact of SDS on mRNA yield and dsRNA is shown in FIG. 11.

TABLE 12
Results of IVT in the presence of SDS

| | | | Yield | dsRNA | |
|---|---|---|---|---|---|
| Sample | SDS concentration | | (mg/mL) | (ppt) | Integrity |
| 1 | 0 M | (0%) | 3.579 | 6.00 | 74.6% |
| 2 | 0.001735 M | (0.05%) | N. D. | N. D. | N. D. |
| 3 | 0.008675 M | (0.25%) | N. D. | N. D. | N. D. |
| 4 | 0.0347 M | (1%) | N. D. | N. D. | N. D. |
| 5 | 0.1388 M | (4%) | N. D. | N. D. | N. D. |

### Example 12. Screening Chaotropic Agent DMSO for dsRNA Formation Reduction

DMSO was tested for the purpose of dsRNA reduction during IVT. DMSO concentrations from 0 M to 1.6 M were evaluated. DMSO did not reduce dsRNA during IVT. Instead, an increase of dsRNA was detected.

The same IVT composition used in Example 1, except chaotropic agent, was applied in the instant example. The results of IVT in the presence of different concentrations of DMSO are shown in Table 13, below. The impact of DMSO on mRNA yield and dsRNA is shown in FIG. 12.

TABLE 13
Results of IVT in the presence of DMSO

| | | | | | dsRNA |
|---|---|---|---|---|---|
| | DMSO | yield | dsRNA | | in DS |
| samples | concentration | (mg/mL) | (ppt) | Integrity | (ppt) |
| 1 | 0 M | 3.579 | 3.00 | 74.6% | 0.79 |
| 2 | 0.4 M | 3.605 | 3.27 | 75.9% | |
| 3 | 0.8 M | 3.450 | 3.30 | 76.2% | |
| 4 | 1.2 M | 2.915 | 3.85 | 76.6% | |
| 5 | 1.6 M | 1.913 | 5.28 | 74.7% | |

### Example 13. Screening Chaotropic Agent Propylene Glycol for dsRNA Formation Reduction

Propylene glycol was tested for the purpose of dsRNA reduction during IVT. Propylene glycol concentrations from 0 M to 1.6 M were evaluated. Propylene glycol did not reduce dsRNA during IVT. Instead, an increase of dsRNA was detected.

The same IVT composition used in Example 1, except chaotropic agent, was applied in the instant example. The results of IVT in the presence of different concentrations of propylene glycol are shown in Table 14, below. The impact of propylene glycol on mRNA yield and dsRNA is shown in FIG. 13.

TABLE 14
Results of IVT in the presence of propylene glycol

| | | | | | dsRNA |
|---|---|---|---|---|---|
| | Propylene glycol | Yield | dsRNA | | in DS |
| Sample | concentration | (mg/mL) | (ppt) | Integrity | (ppt) |
| 1 | 0 M | 3.579 | 2.21 | 74.6% | 0.79 |
| 2 | 0.4 M | 3.133 | 3.16 | 74.9% | |
| 3 | 0.8 M | 2.258 | 4.41 | 77.6% | |
| 4 | 1.2 M | 2.409 | 2.70 | 77.1% | |
| 5 | 1.6 M | 1.588 | 3.89 | 76.8% | |

Among the chaotropic agents tested, urea (at a concentration of 0.1 M to 1.5 M) resulted in a decrease in dsRNA formation; formamide (at a concentration of 0.4 M to 2.8 M) resulted in a decrease in dsRNA formation; sodium perchlorate (at a concentration of 0.01 M to 0.075 M) resulted in a decrease in dsRNA formation; sodium salicylate (at a concentration of 0.005 M to 0.04 M) resulted in a decrease in dsRNA formation; arginine (at a concentration of 0.01 M to 0.075 M) resulted in a decrease in dsRNA formation; and ethanol (at a concentration of 0.4 M to 1.2 M) resulted in a decrease in dsRNA formation. Thiourea (at concentrations of 0.1 M and 0.2 M), 2-propanol (at a concentration of 0.4 M), and n-butanol (at concentrations of 0.16 M and 0.24 M) each resulted in a decrease in dsRNA formation. Other concentrations listed in Table 1, above, did not provide significant reduction in dsRNA formation or shut down the in vitro transcription. Meanwhile, guanidine hydrochloride, DMSO, propylene glycol, and SDS did not significantly reduce the formation of dsRNA, or shut down the in vitro transcription, at the concentrations listed.

### Example 14. Temperature Effect on Urea in Reducing Formation of dsRNA

The effect of temperature on urea's reduction of dsRNA formation was investigated at concentrations of: 0 M (control), 0.4 M, 0.8 M, 1.2 M, and 1.6 M. For these five concentrations, the transcription mixture was incubated at about 34° C., 37° C., and 40° C., each for 2 hrs, followed by DNase I treatment for 20 minutes.

IVT runs with different urea concentrations were performed at 34, 37, and 40° C. The IVT reaction at higher temperature produced less dsRNA, but had lower tolerance for higher urea concentration. For example, 1.6 M urea at 40° C. completely aborted the IVT, whereas a slight product could be seen at 34 and 37° C. On the other hand, higher temperature also accelerated IVT dramatically. More specifically, at 0.8 M urea, IVT performed at 37° C. generated more mRNA than that at 34° C.

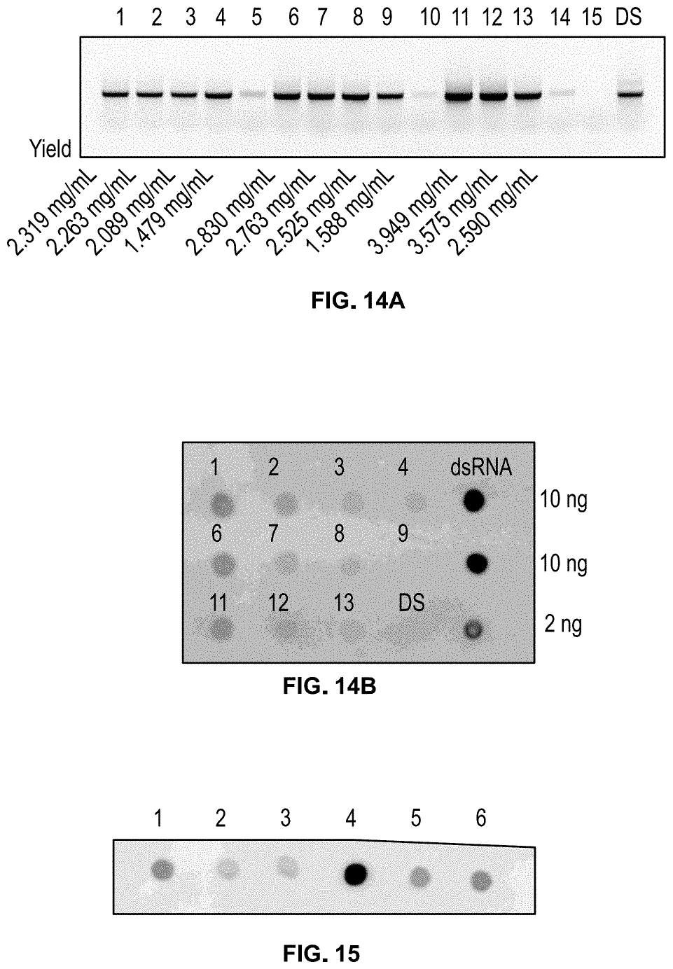

The same IVT composition used in Example 1, except chaotropic agent at different concentrations, was applied in the instant example. The results of IVT in the presence of different concentrations of urea at different temperatures are shown in Table 15, below. The impact of temperature and urea on mRNA yield and dsRNA is shown in FIG. 14.

TABLE 15
Temperature and urea sample information table

| | | |
|---|---|---|
| Lane 1-5 | 0, 0.4, 0.8, 1.2, 1.6 M urea | 34° C., 2 h |
| Lane 6-10 | 0, 0.4, 0.8, 1.2, 1.6 M urea | 37° C., 2 h |
| Lane 11-15 | 0, 0.4, 0.8, 1.2, 1.6 M urea | 40° C., 2 h |

### Example 15. Chaotropic Agent dsRNA Formation Reduction Works on Both Modified NTP and Regular NTP

N1mΨ was used in the study above. To ascertain whether or not the chaotropic agents' relevant activity is affected by NTP chemistry, N1mΨ was compared with regular UTP. dsRNA was significantly reduced by urea during IVT regardless of whether the NTP was modified or regular.

The results of IVT for modified vs. regular NTP in the presence of urea and formamide are shown in Table 16, below. The impact of urea and formamide on IVT involving modified vs. regular NTP is shown in FIG. 15. Per the results, a chaotropic agent can be used to reduce dsRNA formation during IVT to synthesize mRNA using natural nucleotides or modified nucleotides. The ability to use modified NTPs is significant, as immunogenicity can be reduced. This can be especially significant, for example, in the development of vaccines.

TABLE 16
Modified NTP and regular NTP sample information table

| | | | | | dsRNA |
|---|---|---|---|---|---|
| | | Yield | dsRNA | | in DS |
| Sample | Sample information | (mg/mL) | (ppt) | Integrity | (ppt) |
| 1 | N1mΨ used, no chaotropic agents | 3.198 | 5.08 | 79.24% | 1.90 |
| 2 | N1mΨ used, 1 M urea | 2.983 | 2.47 | 78.54% | |
| 3 | N1mΨ used, 1.6 M formamide | 2.937 | 2.21 | 75.48% | |
| 4 | Regular UTP used, no chaotropic agents | 4.477 | 13.30 | 75.38% | |
| 5 | Regular UTP used, 1 M urea | 4.247 | 4.29 | 75.43% | |
| 6 | Regular UTP used, 1.6 M formamide | 4.202 | 4.68 | 75.08% | |

### Example 16. Urea to Reduce Formation of dsRNA in a Scale-Up IVT

To demonstrate the broad application of chaotropic agents for dsRNA reduction during IVT, the IVT process was scaled up to 8 mL and 600 mL, respectively. In each case, the same IVT composition and parameters were used to evaluate urea's dsRNA formation reduction activity at larger scales. IVT was done in water bath.

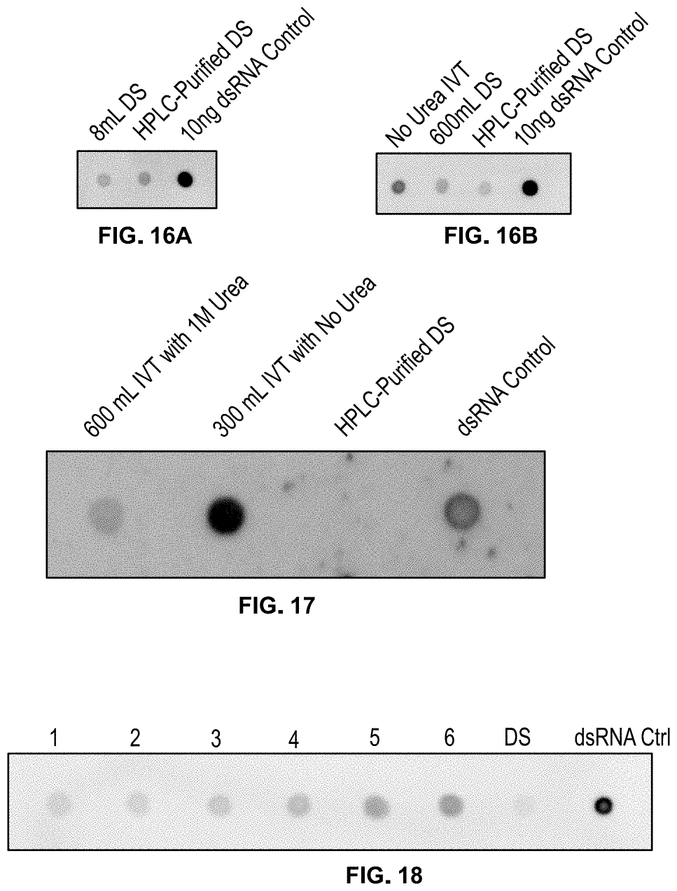

The results of IVT in the presence of urea and scaled up are shown in Table 17, below. The impact of urea on scaled-up IVT is shown in FIG. 16. In certain embodiments, the IVT is scaled up to >600 mL, for example, up to about 1 L, up to about 10 L, up to about 50 L, up to about 100 L, or more.

TABLE 17
Scale-up IVT results in the presence
of urea at different concentrations

| | | mRNA | | | dsRNA |
|---|---|---|---|---|---|
| IVT Volume | Urea Conc. | yield | dsRNA | | in DS |
| (mL) | (M) | (mg/mL) | (ppt) | Integrity | (ppt) |
| 8 | 1.0 | 3.56 | 0.60 | 85.4% | 0.70 |
| 600 | 1.0 | 3.51 | 1.59 | 83.57% | 1.08 |

### Example 17. Urea to Reduce Formation of dsRNA in a Scale-Up IVT in a Bioreactor

To further demonstrate the application of chaotropic agents to reduce the formation of dsRNA independent of IVT scales, 600 mL IVT was performed in a more scalable wave reactor with a 2 L disposable sterile wave bag. Natural UTP and 1M urea were used, but no capping reagent was included in IVT. This also indicated that chaotropic agent to reduce formation of dsRNA during IVT was independent of capping method (co-transcriptional capping agent, or enzymatic capping, or no capping). The produced 5′-ppp mRNA was suitable for enzymatic capping (though not done in the instant example). Control sample (300 mL IVT with no urea) was done in the wave reactor too.

The results of IVT in the presence of urea and scaled up in a bioreactor are shown in Table 18, below. The impact of urea on scaled-up IVT in a bioreactor is shown in FIG. 17. In certain embodiments, the IVT is scaled up to >600 mL, for example, up to about 1 L, up to about 10 L, up to about 50 L, up to about 100 L, or more.

TABLE 18
Bioreactor scale-up IVT results in the presence
of urea at different concentrations

| IVT | Urea | mRNA | | mRNA integrity | dsRNA |
|---|---|---|---|---|---|
| Volume | Conc. | yield | dsRNA | prior to | in DS |
| (mL) | (M) | (mg/mL) | (ppt) | purification | (ppt) |
| 300 | 0 | 4.13 | 26.9 | 82.1% | Unable to |
| 600 | 1.0 | 3.86 | 3.45 | 78.1% | determine |

### Example 18. Delayed Addition of Chaotropic Agent to Control the Levels of dsRNA

In certain embodiments of the disclosure, chaotropic agents can be added to the IVT reaction mixture at a later time. Thus potentially controlling the level of dsRNA could be beneficial, for example, when a certain level of immunogenicity is desired, for example, for mRNA vaccines. In the instant study, urea solution was added into a standard IVT mixture at an interval of 30 minutes, the amount of dsRNA was increased when urea was added at a later time point.

The same IVT composition as used in Example 1, with 1 M urea, was applied in this example. All transcriptions were kept at 37° C. for a total of 180 minutes, and all results were measured at the end of transcription. The results of IVT in the presence of urea added at different time points are shown in Table 19, below. The impact of adding urea at different time points on dsRNA is shown in FIG. 18.

TABLE 19
Addition of urea into IVT mixture at different time points

| | | | | | dsRNA |
|---|---|---|---|---|---|
| | | Yield | dsRNA | | in DS |
| Samples | addition time | (mg/mL) | (ppt) | integrity | (ppt) |
| 1 | 0 min, urea added | 2.77 | 1.70 | 70.7% | 1.00 |
| 2 | 30 min, urea added | 3.09 | 1.40 | 71.9% | |
| 3 | 60 min, urea added | 3.47 | 2.19 | 72.3% | |
| 4 | 90 min, urea added | 3.60 | 3.38 | 70.1% | |
| 5 | 120 min, urea added | 3.93 | 4.79 | 71.5% | |
| 6 | 120 min, water added | 4.30 | 5.36 | 71.8% | |

### Example 19. Using More than One Chaotropic Agent at the Same Time to Reduce Formation of dsRNA

In some embodiments of the disclosure, more than one chaotropic agent is used to reduce dsRNA formation during IVT. In the instant study, a combination of two chaotropic agents was tested. The results indicated that dsRNA was effectively reduced during IVT.

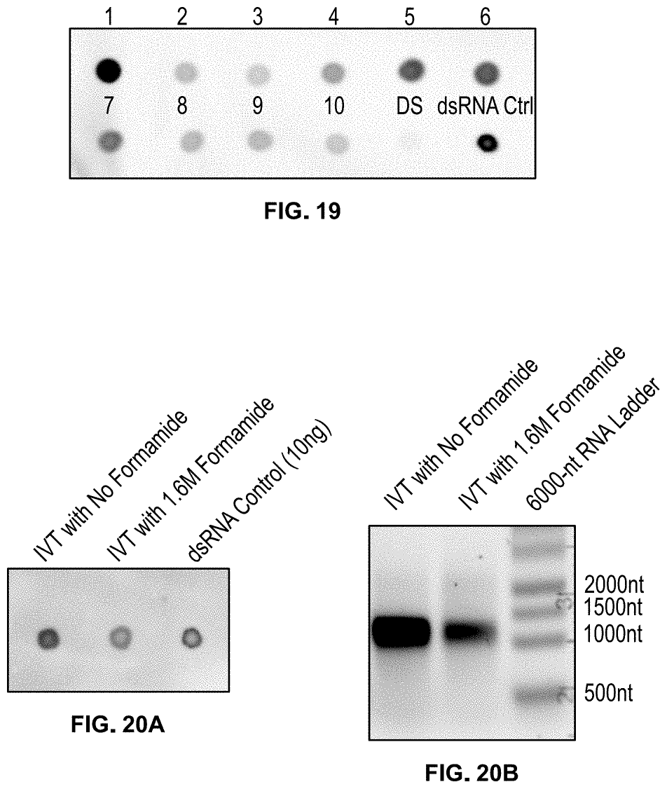

The same IVT composition used in Example 1, except chaotropic agent(s) and N1mΨ, was applied in the instant example to study the use of more than one chaotropic agent to reduce formation of dsRNA. Natural UTP was used in this example instead of N1mΨ. The results of IVT in the presence of more than one chaotropic agent are shown in Table 20, below. The impact of using more than one chaotropic agent on dsRNA is shown in FIG. 19.

TABLE 20
Use of more than one chaotropic agent to reduce formation of dsRNA

| | | | | | dsRNA |
|---|---|---|---|---|---|
| | | Yield | dsRNA | | in DS |
| Samples | Chaotropic agents | (mg/mL) | (ppt) | Integrity | (ppt) |
| 1 | None | 4.11 | 15.97 | 69.1% | 0.33 |
| 2 | 1M urea | 2.91 | 2.92 | 69.5% | |
| 3 | 1.6 M formamide | 3.04 | 2.22 | 70.4% | |
| 4 | 50 mM NaClO₄ | 4.22 | 4.41 | 72.1% | |
| 5 | 0.5 M urea | 4.23 | 9.84 | 72.8% | |
| 6 | 0.8 M formamide | 3.97 | 9.95 | 73.2% | |
| 7 | 25 mM NaClO₄ | 4.99 | 6.25 | 71.2% | |
| 8 | 0.5 M urea + 0.8 M formamide | 3.40 | 3.02 | 71.7% | |
| 9 | 0.5 M urea + 25 mM NaClO₄ | 4.56 | 3.47 | 71.0% | |
| 10 | 0.8 M formamide + 25 mM | 4.87 | 2.31 | 70.8% | |
| | NaClO₄ | | | | |

### Example 20. Formamide to Reduce Formation of dsRNA in IVT with a Template Encoding ˜1300-Nt RNA

To demonstrate the broad application of chaotropic agents in reducing formation of dsRNA during IVT, the DNA template was extended to a different DNA template that encodes an about 1300-nt EGFP-related RNA. Regular NTPs were used for IVT, and 5′ cap1 was obtained through post-transcriptional enzymatic capping. 1.6 M formamide was used to reduce dsRNA. After spin column purification, the mRNA was subjected to vaccinia capping system to obtain Cap1 structure.

Vaccinia capping enzyme and 2′-O-methyltransferase were used to obtain Cap1 structure for the mRNA in the presence of 0.5 mM GTP and 0.2 mM SAM. mRNA was heated at 65° C. for 5 minutes and cooled on ice for 5 minutes followed by adding buffer, GTP, SAM, and the two enzymes. The reaction was incubated at 37° C. for 60 minutes, and the mRNA was purified by spin column. The results of IVT with a plasmid encoding a 1300 nt RNA in the presence of chaotropic agent formamide with respect to dsRNA formation and mRNA yield are shown in FIG. 20.

Thus, numerous chaotropic agents were screened for their reduction of formation of dsRNA during IVT. Select chaotropic agents including urea and formamide additionally showed efficacy in scale-up IVT, IVT with regular NTP vs. modified NTP, IVT with different DNA templates, and IVT at different temperatures.

While chaotropic agents reduce formation of dsRNA during IVT by partially breaking down unwanted intermolecular or intramolecular nucleic acid interactions believed to be the causes of dsRNA formation during IVT, they can also denature enzymes and shut down IVT partially or completely, resulting in low or no mRNA yield. In certain embodiments, non-charged chaotropic agents such as urea and formamide are employed to reduce formation of dsRNA during IVT.

It is demonstrated herein that adding chaotropic agents into the IVT reaction is a simple and effective way to reduce the formation of dsRNA. Furthermore, the claimed methods and compositions may replace other existing dsRNA-removal technologies, such as RP-HPLC and CHT chromatography processes, and achieve higher mRNA integrity, resulting in potent and low immuno-stimulatory mRNA. Of note, the claimed methods and compositions greatly simplify the mRNA production process by reducing or entirely removing its reliance on purification methods, e.g., chromatography steps, for dsRNA reduction, in turn, eliminating scalability concerns. Therefore, the claimed methods and compositions significantly drive the production of mRNA therapeutics.

The present disclosure is not to be limited in scope by the specific embodiments described herein. Indeed, various modifications of the disclosure in addition to those described herein will become apparent to those skilled in the art from the foregoing description and the accompanying figures. Such modifications are intended to fall within the scope of the appended claims. The above specification, examples and data provide a complete description of the composition, manufacture of the composition and use of the composition of the invention. Since many embodiments of the invention can be made without departing from the spirit and scope of the invention, the invention resides in the claims hereinafter appended.

What is claimed is:

1. A method of reducing, minimizing, or inhibiting the formation of double-stranded ribonucleic acid (dsRNA) during in vitro transcription, comprising adding at least one chaotropic agent to an in vitro transcription reaction mixture.

2. A method of reducing, minimizing, or inhibiting intramolecular base-pairing within a ribonucleic acid (RNA) transcript and/or intermolecular base-pairing between an RNA transcript and deoxyribonucleic acid (DNA) or another RNA during in vitro transcription, comprising adding at least one chaotropic agent to an in vitro transcription reaction mixture.

3. The method of claim 1 or 2, wherein the transcription yields ribonucleic acid (RNA).

4. The method of claim 2 or 3, wherein the RNA is messenger RNA (mRNA).

5. The method of any one of claims 2 to 4, wherein the amount or yield of RNA is not significantly reduced by the addition of the at least one chaotropic agent.

6. The method of any one of claims 1 to 5, wherein the at least one chaotropic agent is selected from the group consisting of urea, formamide, sodium salicylate, ethanol, sodium perchlorate, arginine, n-butanol, thiourea, and 2-propanol.

7. The method of claim 6, wherein the at least one chaotropic agent is urea.

8. The method of claim 7, wherein the urea is at a concentration of from about 0.1M to about 1.6M.

9. The method of claim 6, wherein the at least one chaotropic agent is formamide.

10. The method of claim 9, wherein the formamide is at a concentration of from about 0.1M to about 2.8M.

11. The method of claim 6, wherein the at least one chaotropic agent is sodium perchlorate.

12. The method of claim 11, wherein the sodium perchlorate is at a concentration of from about 0.01M to less than about 0.15M.

13. The method of claim 6, wherein the at least one chaotropic agent is sodium salicylate.

14. The method of claim 13, wherein the sodium salicylate is at a concentration of from about 0.005M to less than about 0.1M.

15. The method of claim 6, wherein the at least one chaotropic agent is arginine.

16. The method of claim 15, wherein the arginine is at a concentration of from about 0.01M to less than about 0.2M.

17. The method of claim 6, wherein the at least one chaotropic agent is ethanol.

18. The method of claim 17, wherein the ethanol is at a concentration of from about 0.4M to less than about 1.6M.

19. The method of claim 6, wherein the at least one chaotropic agent is thiourea.

20. The method of claim 19, wherein the thiourea is at a concentration of from about 0.1M to about 0.2M.

21. The method of claim 6, wherein the at least one chaotropic agent is 2-propanol.

22. The method of claim 21, wherein the 2-propanol is at a concentration of about 0.4M.

23. The method of claim 6, wherein the at least one chaotropic agent is n-butanol.

24. The method of claim 23, wherein the n-butanol is at a concentration of from about 0.16M to about 0.24M.

25. The method of any one of the preceding claims, wherein the reaction mixture is on the order of microliters to the order of liters.

26. The method of any one of the preceding claims, wherein the presence or absence of dsRNA is determined using denaturing gel electrophoresis, native gel electrophoresis, anti-dsRNA antibody, intact mass spectrometry, and/or controls for dsRNA.

27. A composition comprising RNA prepared according to the method of any one of the preceding claims, wherein the composition is substantially free of dsRNA.

28. An in vitro transcription reaction mixture comprising at least one chaotropic agent for use in the preparation of ribonucleic acid (RNA).

29. The reaction mixture of claim 28, wherein the RNA is substantially free of double-stranded ribonucleic acids (dsRNA).

30. The reaction mixture of claim 28 or 29, wherein the RNA is messenger RNA (mRNA).

31. The reaction mixture of any one of claims 28-30, wherein the at least one chaotropic agent is selected from the group consisting of urea, formamide, sodium salicylate, ethanol, sodium perchlorate, arginine, n-butanol, thiourea, and 2-propanol.

32. The reaction mixture of claim 31, wherein the at least one chaotropic agent is urea.

33. The reaction mixture of claim 32, wherein the urea is at a concentration of from about 0.1M to about 1.6M.

34. The reaction mixture of claim 31, wherein the at least one chaotropic agent is formamide.

35. The reaction mixture of claim 34, wherein the formamide is at a concentration of from about 0.1M to about 2.8M.

36. The reaction mixture of claim 31, wherein the at least one chaotropic agent is sodium perchlorate.

37. The reaction mixture of claim 36, wherein the sodium perchlorate is at a concentration of from about 0.01M to less than about 0.15M.

38. The reaction mixture of claim 31, wherein the at least one chaotropic agent is sodium salicylate.

39. The reaction mixture of claim 38, wherein the sodium salicylate is at a concentration of from about 0.005M to less than about 0.1M.

40. The reaction mixture of claim 31, wherein the at least one chaotropic agent is arginine.

41. The reaction mixture of claim 40, wherein the arginine is at a concentration of from about 0.01M to less than about 0.2M.

42. The reaction mixture of claim 31, wherein the at least one chaotropic agent is ethanol.

43. The reaction mixture of claim 42, wherein the ethanol is at a concentration of from about 0.4M to less than about 1.6M.

44. The reaction mixture of claim 31, wherein the at least one chaotropic agent is thiourea.

45. The reaction mixture of claim 44, wherein the thiourea is at a concentration of from about 0.1M to about 0.2M.

46. The reaction mixture of claim 31, wherein the at least one chaotropic agent is 2-propanol.

47. The reaction mixture of claim 46, wherein the 2-propanol is at a concentration of about 0.4M.

48. The reaction mixture of claim 31, wherein the at least one chaotropic agent is n-butanol.

49. The reaction mixture of claim 48, wherein the n-butanol is at a concentration of from about 0.16M to about 0.24M.

50. The composition of claim 27, wherein the presence or absence of dsRNA is determined using denaturing gel electrophoresis, native gel electrophoresis, anti-dsRNA antibody, intact mass spectrometry, and/or controls for dsRNA.

51. The reaction mixture of any one of claims 28 to 49, wherein the presence or absence of dsRNA is determined using denaturing gel electrophoresis, native gel electrophoresis, anti-dsRNA antibody, intact mass spectrometry, and/or controls for dsRNA.

52. A method of reducing, minimizing, or inhibiting the formation of double-stranded ribonucleic acid (dsRNA) during the preparation of ribonucleic acid (RNA), comprising adding at least one chaotropic agent to a starting reaction mixture.

53. A method of reducing, minimizing, or inhibiting intramolecular base-pairing within a ribonucleic acid (RNA) transcript and/or intermolecular base-pairing between an RNA transcript and deoxyribonucleic acid (DNA) or another RNA during the preparation of ribonucleic acid (RNA), comprising adding at least one chaotropic agent to a starting reaction mixture.
